(19) United States
(12) Patent Application Publication
(10) Pub. No.: US 2003/0224446 A1
(43) Pub. Date: Dec. 4, 2003

(54) Method of predicting cytokine response to tissue injury

(76) Inventors: G. Jean Harry, Chapel Hill, NC (US); Alessandra Bruccoleri, Corsico (IT); Donald Schmechel, Durham, NC (US)

Correspondence Address: DYKEMA GOSSETT PLLC, 39577 WOODWARD AVENUE, SUITE 300, BLOOMFIELD HILLS, MI, 48304-5086, US

(21) Appl. No.: 10/324,374

(22) Filed: Dec. 19, 2002

Related U.S. Application Data
(60) Provisional application No. 60/342,567, filed on Dec. 20, 2001.

(51) Int. Cl. G01N 33/53

(52) U.S. Cl. 435/7.1

(57) ABSTRACT

A method of predicting cytokine production in response to tissue injury by assaying for APOE

### RELATED APPLICATION

This application claims priority from U.S. provisional application Serial No. 60/342,567 filed Dec. 20, 2001.

### FIELD OF THE INVENTION

The present invention relates generally to the in vivo production of cytokines in response to tissue injuries and, more specifically, to a method of predicting cytokine response to tissue injury.

### BACKGROUND OF THE INVENTION

It is known that cytokines make up a diverse population of soluble and membrane-bound proteins that regulate immune and inflammatory responses that can have both positive and negative effects. Their influence and effects reach beyond the immune system and have the capability to signal physiological and pathological processes expressed in most cells in the body, including those in the nervous system.

The pathogenesis of Alzheimer's disease (AD) is characterized by neural injury that includes a sequence of cellular events leading to the formation of neurofibrillary tangles, neuritic plaques, and loss of synapses. In addition to the neuronal component, there is evidence of a role for “inflammatory” responses in the pathogenesis of AD. These responses are marked by astrocyte reactivity and microglia activation throughout the cortex. In particular, activated microglia are found to be clustered near amyloid plaques that contain other components such as β-amyloid protein, thrombin, and apolipoprotein E. This data, in conjunction with the identified features of microglia as a macrophage like cell within the brain, have led to the supposition that microglia and their immune like secreted factors play a prominent role in the onset and progression of disease related neurodegeneration.

Microglia respond to trauma normally by an increase in number, migration, and altered morphology and immunophenotype. In an inflammatory response, microglia present antigen to immune cells and secrete proinflammatory cytokines such as tumor necrosis factor (TNF), interleukin-1 (IL-1), and nitric oxide, resulting in cytotoxicity and tissue damage. In addition, stimulated microglia respond to interleukin-1β, tumor necrosis factor-α, and y-interferon. There is increasing evidence for cytokines playing a key role in various neurodegenerative diseases, including AD.

A variety of factors and processes have been implicated in the initiation and progression of AD pathology. Such factors include amyloid fragment deposition, reactive gliosis, α-1 antichymotrypsin, and apolipoprotein E (apoE). The various genetic risk factors for AD and the subsequent animal models have provided a great deal of information regarding the pathogenesis and associated factors for such neurodegenerative disorders. In humans, three common apoE alleles (apoE2, E3, and E4) exist with varying degrees of risk conferred by each. The allele 4 specifically shows an association with decreased longevity, increased low density lipoprotein (LDL) levels, increased risk for coronary vascular disease, and increased rick for AD. It has further been established that there is a change in allele frequence of TNFβ in AD patients compared to those individuals not suffering from AD. Tarkowski E. et al., TNF gene polymorphism and its relation to intracerebral production of TNFα and TNFβ in AD, Neurology. 54(11):207781, Jun. 13, 2000.

Accordingly, since cytokines can produce cytotoxicity and tissue damage it would be desirable to predict the cytokine response of a particular subject to neural injury.

### SUMMARY OF THE INVENTION

In one aspect, the present invention provides a method of predicting the levels of certain cytokines produced in response to tissue injury, including neural injury of the type associated with Alzheimer's disease, by determining APOE genotype and the age of the subject. In another aspect, the present invention provides a method of predicting whether a tissue injury will elevate TNFβ in a subject.

### BRIEF DESCRIPTION OF THE DRAWINGS

FIG. 1 is a hematoxylin and eosin staining of hippocampal dentate granule cells in (A) saline-injected wild-type and (B) TMT-exposed wild-type, (C) APOE knockout, and (D) APOE4 mice. Mice receive an ip injection of saline (control) or TMT hydroxide (2 mg/kg body wt) 24 h prior to collection of tissue. Severe neuronal necrosis was characterized by nuclear pyknosis and karyolysis in the dentate granule cells (arrow) with no degeneration of the pyramidal neurons (open arrow). Magnification, ×100.

FIG. 2 is a hematoxylin and eosin staining and GFAP immunoreactivity in the hippocampus 24 h following acute intraperitoneal injection of either saline or TMT hydroxide (2.0 mg/kg body weight). Top panels demonstrate the basal level of cellularity by H&E staining (A) and astrocyte GFAP immunoreactivity (B) in all animals following injection of saline. GFAP immunoreactivity induced by TMT in wild-type C and D), APOE knockout (E and F), and APOE4 (G and H) mice. Astrocytes were evident throughout the hippocampus and displayed thick cell bodies and processes characteristic of astrocyte reactivity following TMT as compared with the thin processes evident in the same region following saline (B). Magnification, ×350.

FIG. 3 is relative mRNA levels of ICAM-1, A-20, Mac-1, EB-22, and GFAP in the hippocampus 24 h following acute ip injection of either saline or TMT hydroxide (2.0 mg/kg body wt) to 21-day-old wild-type, APOE knockout, and APOE4 male mice. Values represent the mean volumetric units for each gene transcript relative to volumetric units of transcript for L32 for four animals per group. The ^{a} designates significant difference from vehicle control; ^{b} designates significant difference from wild type as determined by individual one-way ANOVAs for each genotype following a 2×3 ANOVA (p<0.05).

FIG. 4 is relative mRNA levels of ICAM-1, A-20, Mac-1, EB-22, and GFAP in the hippocampus 24 h following acute ip injection of either saline or TMT hydroxide (2.0 mg/kg body wt) to 8-month-old wild type, APOE knockout, and APOE4 male mice. Values represent the mean volumetric units for each gene transcript relative to volumetric units of transcript for L32 for four animals per group. The a designates significant difference from vehicle control; ^{b} designates significant difference from wild type as determined by individual one-way ANOVA for each genotype following a 2×3 ANOVA (p<0.05).

FIG. 5 is relative mRNA levels of TNFβ, TNFα, IL-1α, MIP-1α, and TGF-β1 in the hippocampus 24 h following acute ip injection of either saline or TMT hydroxide (2.0 mg/kg body wt) to 21-day-old wild-type, APOE knockout, and APOE4 male mice. Values represent the mean volumetric units for each gene transcript relative to volumetric units of transcript for L32 for five animals per group. The a designates significant difference from vehicle control; ^{b} designates significant difference from wild type as determined by individual one-way ANOVA for each genotype following a 2×3 ANOVA (p<0.05).

FIG. 6 is relative mRNA levels of TNFβ, TNFα, IL-1α, MIP-1α, and TGF-β1 in the hippocampus 24 h following acute ip injection of either saline or TMT hydroxide (2.0 mg/kg body wt) to 8-month-old wild-type, APOE knockout, and APOE4 male mice. Values represent the mean volumetric units for each gene transcript relative to volumetric units of transcript for L32 for four animals per group. The ^{a} designates significant difference from vehicle control; ^{b} designates significant difference from wild type as determined by individual one-way ANOVA for each genotype following a 2×3 ANOVA (p<0.05).

FIG. 7 is relative mRNA levels of TNFβ, TNFα, IL-1α, MIP-1α, and TGF-β1 in the cortex 24 h following acute ip injection of either saline or TMT hydroxide (2.0 mg/kg body wt) to 8-month-old wild-type, APOE knockout, and APOE4 male mice. Values represent the mean volumetric units for each gene transcript relative to volumetric units of transcript for L32 for four animals per group. The a designates significant difference from vehicle control (p<0.05) as determined by individual one-way ANOVA for each genotype following a 2×3 ANOVA.

FIG. 8 is relative mRNA levels of TNFβ, TNFβ, IL-1α, MIP-1α, and TGF-β1 in the cerebellum 24 h following acute ip injection of either saline or TMT hydroxide (2.0 mg/kg body wt) to 8-month-old wild-type, APOE knockout, and APOE4 male mice. Values represent the mean volumetric units for each gene transcript relative to volumetric units of transcript for L32 for three animals per group. The ^{a} designates significant difference from vehicle control (p<0.05) as determined by individual one-way ANOVA for each genotype following a 2×3 ANOVA.

### DETAILED DESCRIPTION OF THE PREFERRED EMBODIMENTS

The present invention relates to have discovery that the production of TNFβ is a function of APOE genotype; more specifically, the complete absence (or inactivation) of APOE genes results in down regulation of TNFβ. The present inventors have also discovered that the influence of APOE genotype on initial injury response is age-dependent. In particular it has been found that the presence of both APOE allele 4 (APOE4) and advanced age of the subject result in greater cytokine response for TNFβ, TNFα, GFAP and MIP-1α to neural injury than young subjects with the APOE4 genotype.

The current study used a chemically induced injury model to examine the differential susceptibility of APOE knockout mice and mice containing the human APOE allele 4 transgene. In mice, systemic administration of trimethyltin (TMT) produces a distinct pattern of neuronal necrosis in hippocampal dentate granule cells accompanied by astrogliosis and microglia activation and activation of the proinflammatory cytokine cascase. Using this model of chemical injury, the present inventors have shown that the influence of APOE genotype on the initial injury response is age dependent. In young, 21-day-old mice, the basal mRNA levels for proinflammatory cytokines were similar across all genotypes and similar elevations were seen following TMT in the host-response genes, initiation of the cytokine cascade, and in morphological responses of neuronal cell death and astrocytes. Examination of mice at 8 months of age demonstrated higher mRNA levels for GFAP, TNFβ, MIP-1α, TGF-β1 in both APOE knockout and APOE4 mice as compared to wild-type mice. Following TMT, APOE knockout mice showed a distinct lack of elevation in mRNA levels for TNFα and less elevation in macrophage inflammatory protein-1α (MIP-1α) mRNA as compared to the response seen in wild-type mice.

APOE knockout mice and human apolipoprotein E4 isoform-specific transgencic mice were generated as previously described (Xu et al., 1998). The APOE knockout mice carry an inactivated endogenous APOE gene disrupted by gene targeting in embryonic stem cells (Piedrahita et al., 1992) and were generated as previously described (Xu et al., 1998). Human APOE4 allele-specific mice were generated by microinjection of human genomic DNA fragments and isolated and purified from lymphoblasts of individuals verified to be homozygous carriers for the APOE4 allele into single-celled embryos fertilized by APOE knockout males to produce transgenic mice (Xu et al., 1996). Normal dams were a cross of mouse strains C57BL/6J×DBA/2J (B6D2F1). Male founder mice were backbred to female APOE knockout mice to produce animals hemizygous for human APOE allele 4 transgene and homozygous for inactivated mouse APOE gene. Mice were bred to homozygosity and backbred further into APOE knockout strains on B6 background (Jackson Laboratories, Bar Harbor, Me.) to remove genetic influence of DBA/2J background strain. Confirmation of genotype was conducted by PCR on tail DNA as previously described (Xu et al., 1996; 1998).

Male mice were housed in an animal facility at a constant temperature (21°±2° C.) and humidity (50%±5%) and on a 12-h light/dark cycle. Food and water were available ad libitum. At either 21 days or 8 months of age, APOE knockout mice, APOE4 mice, and wild-type (C57BL6J) controls were randomly assigned to experimental groups and administered a single dose of either trimethyltin hydroxide (TMT 2.0 mg/kg bwt, i.p.; Alpha Products, Danvers, Mass.) or saline vehicle in a dosing volume of 4 ml/kg body weight. All experiments were conducted in compliance with an animal protocol approved by the NIEHS/NIH Animal Care and Use Committee.

Based upon previous studies with TMT exposure (Bruccoleri et al., 1998, 1999), a minimum of six mice per group were examined for morphological changes in the hippocampus 24 h following injection. Animals were lightly anesthetized with CO₂ and decapitated and brains were removed from the cranium, bisected in the midsagittal plane, and immersion-fixed in 4% paragormaldehyde/0.1 M phosphate-buffered saline (PBS; pH 7.2) overnight at room temperature. Eight-micron paraffin-embedded sections were stained by routine hematoxylin and eosin (H&E) to visualize tissue cellularity. Astrocytes were identified by immunohistochemistry using polyclonal glial fibrillary acidic protein (GFAP) antibodies (Dako Corp., Carpinteria, Calif.). Briefly, rehydrated sections were treated with 3% H₂O₂, subjected to heat-induced epitope retrieval (HIER) using a decloaking chamber (Biocare Medical, Walnut Creek, Calif.), and incubated with nonimmune goat serum (1.5:100 dilution in 1% BSA/PBS) prior to incubation with rabbit anti-rat GFAP (1:2000 in 1:1 solution of 1% BSA and 1% powered milk in PBS). Sections were incubated with a secondary IgG antibody followed by ABC (avidin-biotin complex) reagent (Vectastain Elite, Vector Laboratories, Burlingame, Calif.) and detected by 3,3′-diaminobenzidine tetrahydrochloride (DAB) substrate (Sigma-Aldrich Research, St. Louis, Mo.). Microglia cells were identified by lectin binding (Bandeiraea Simplicifolia BS-4, 1:10; Sigma Chemical Co.; St. Louis, Mo.) by the method of Streit and Kreutzberg (1987) using HIER. Sites containing microglia bound peroxidase-lectin conjugates were visualized by DAB-H₂O₂ substrate containing CO₂ ions.

Based upon earlier studies with TMT exposure (Bruccoleri et al., 1998, 1999), Rnase protection assays were conducted at 24-h post-TMT. Multiprobe sets were used for simultaneous detection of mRNAs as previously described (Hobbs, 1993; Bruccoleri et al., 1999). The first set, provided by Dr. Iain Campbell (Scripps Research Institute, San Diego, Calif.), contained probes for intercellular adhesion molecule-1 (ICAM-1), inducible nitric oxide synthase (iNOS), an anti-apoptotic TNFα-inducible early response gene (A20), macrophase-1 antigen (Mac-1), a murine acute-phase response gene homologous to the α-1-antichymotrypsin gene (EB22/5), and GFAP as previously described (Campbell et al., 1994). The second set was purchased from Pharmingen (San Diego, Calif.) and contained probes for tumor necrosis factor-β (TNFβ) and -α (TNFα), interleukin-1α (IL-1α), macrophage inflammatory protein-1α (MIP-1α), and transforming growth factor-β1 (TGF-β1). Each probe set contained probes for L32 and GAPDH. A minimum of 4 animals per group was used in each experiment. Individual fragments were separated by 5% acrylamide/8 M urea sequencing gel electrophoresis and visualized by autoradiography (Hyperfilm-MP film; Amersham, Buckinghamshire, U.K.). Radioactivity in each fragment was visualized by phosphorimager (Molecular Dynamics, Sunnyvale, Calif.) and relative volume determined using Imagequant (Molecular Dynamics) and normalized relative to the corresponding L32 volume.

At each age, 21 days or 8 months, data for each mRNA transcript displayed a homogenous distribution of variance, as determined by Bartlett's test for homogeneity of variance, and were analyzed by a 2×3 ANOVA with genetic background and exposure as factors using the SPSS statistical package. Subsequent independent group mean comparisons between each saline-injected genotype or between TMT and saline-injected mice, within each genotype, were conducted using individual one-way ANOVAs for each gene transcript. A statistical significance level was set at p<0.05.

Within 24 h of TMT dosing, all mice displayed characteristic features of whole-body tremor in the absence of seizure activity. In the young animals, 21 days of age, survival rate was slightly decreased in both the APOE knockout (10%) and the APOE4 mice (20%). In both genetically altered mice, the severity of tremor induced by TMT was slightly greater than that seen in the wild-type mice. No signs of aggressiveness or seizures were evidence in any of the mide exposed to TMT. At 8 months of age, APOE4 mice demonstrated an increased sensitivity to the systemic toxicity of TMT with a decrease in survival rate of approximately (40%) as compared to both APOE knockout and wild-type mice. This may not be related to a nervous system effect but rather to a generalized increased sensitivity to the systemic toxicity of TMT. Other studies have demonstrated an increase susceptibility to the toxicity of endotoxemia and Klebsiella pneumoniae (deBont et al., 1999) and Listeria monocytogenes (Roselaar & Daugherty, 1998) in APOE knockout mice.

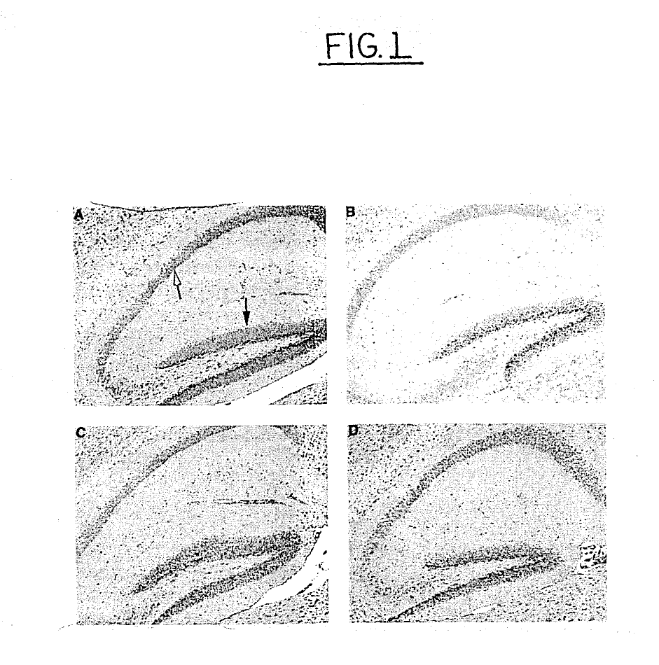

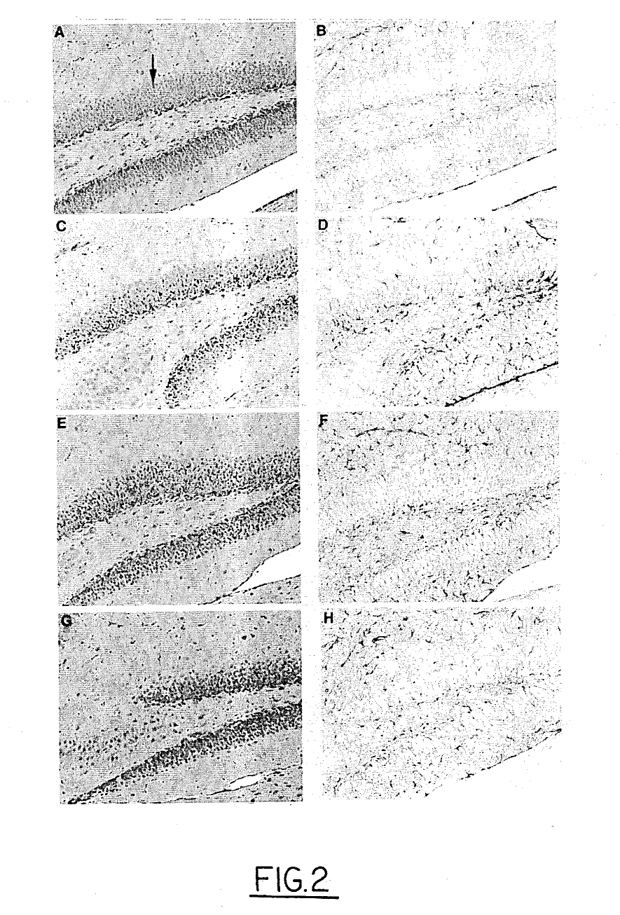

Twenty-four hours following exposure to TMT, neuronal cell death was evident in the hippocampus of wild-type mice as compared to normal cell morphology in the saline-injected mice (FIG. 1A). Cell death, following TMT, was characterized by nuclear pyknosis and karyolysis in dentate granule cells of the hippocampus (FIG. 1B). Neurons of the pyramidal cell layer remained intact with no indication of neuronal cell death (FIG. 1B). Both APOE knockout (FIG. 1C) and APOE4 (FIG. 1D) mice showed a similar pattern and level of severity of neuronal damage in the dentate and no loss of pyramidal neurons. Higher magnification of H&E-stained sections showed characteristic nuclear pyknosis and karyolysis in the dentate granule cells of the hippocampus in wild-type (FIG. 2C), APOE knockout (FIG. 2E), and APOE4 (FIG. 2G) mice as compared to normal neuronal integrity seen in all saline-dosed mice (FIG. 2A). Wild-type mice showed a relatively high background staining for GFAP (FIG. 2B) with little change following TMT (FIG. 2D). APOE knockout and APOE4 mice showed similar basal levels of GFAP immunostaining as compared to wild type (data not shown). Following TMT, a slight increase in GFAP immunoreactivity was seen in astrocytes throughout the hippocampus and at site of neuronal injury in both APOE knockout (FIG. 2F) and APOE4 mice (FIG. 2H).

It has been previously reported (Bruccoleri et al., 1998) that microglia cells in the hippocampus first demonstrate a morphological response by 72 h following TMT dosing. In order to determine if alterations in APOE would result in an early activation of microglia celia, lectin staining was conducted on samples at the 24-h time point. Consistent with these previous results, lectin histochemistry did not detect reactive/activated microglia in the hippocampus at 24 h post-TMT in the wild-type mice, the APOE knockout mice, or the APOE4 mice.

At 21 days of age, no significant differences between genotypes were seen in mRNA levels for ICAM-1, A20, or Mac-1. However, APOE4 mice displayed significantly higher (p<0.05) levels of GFAP and EB22 mRNA as compared to both wild-type and APOE knockout mice (FIG. 3). TMT exposure produced a slight but significant elevation (p<0.05) in ICAM-1 mRNA levels in all mice examined (FIG. 3). ICAM-1 has been demonstrated to serve an accessory function in immune activation with the assistance in trafficking of microglia to the site of injury (Butcher, 1990). This slight elevation suggests that ICAM-1 is providing an accessory function in TMT-induced pathogenesis. Following TMT, both wild-type and APOE knockout mice showed a significant (p<0.05) elevation of EB22 mRNA levels (FIG. 3). APOE4 mice, however, showed a slightly higher level of EB22 but no elevation following TMT (FIG. 3). GFAP mRNA levels were significantly elevated in both wild-type and APOE knockout mice exposed to TMT (FIG. 3). mRNA levels for Mac-1, A20, and iNOS were not altered by exposure to TMT in any group of mice examined.

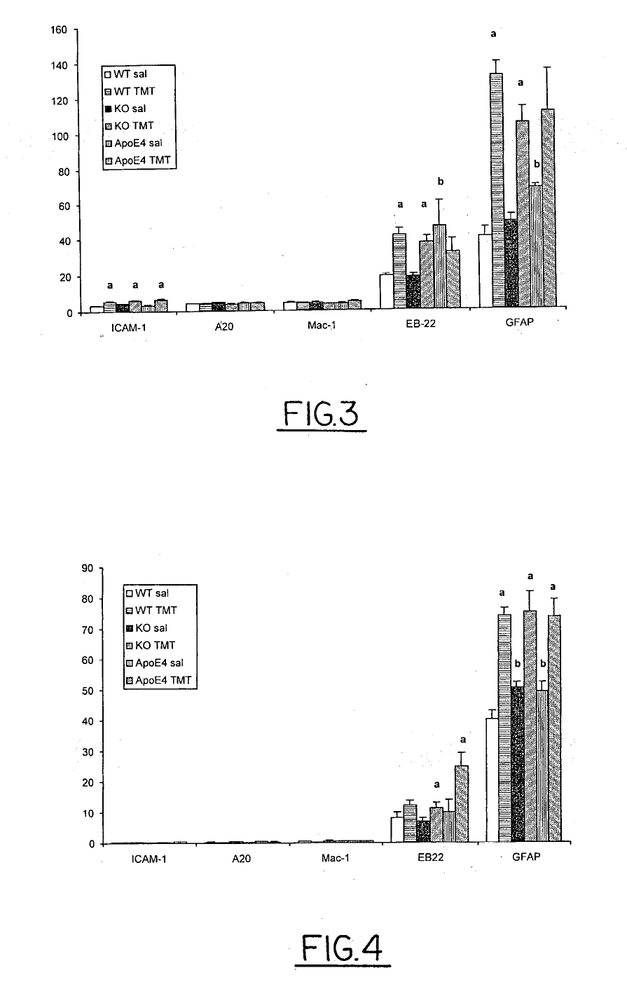

In 8-month-old mice, no significant differences were seen between wild-type and both APOE genotype mice for ICAM-1, A20, Mac-1, and, in contrast to a previous report (Liscastro et al., 1999), EB22 (FIG. 4). GFAP mRNA levels were significantly elevated in both APOE knockout and APOE4 mice relative to wild-type mice (FIG. 4). At 24 h postdosing, TMT produced an elevation of EB22 mRNA levels in the hippocampus in the APOE knockout and APOE4 mice. No elevation was seen in the wild-type mice. TMT exposure resulted in an elevation in GFAP mRNA levels in all groups of mice (FIG. 4). mRNA levels for Mac-1, A20, and iNOS were not altered by exposure to TMT in any group of mice examined.

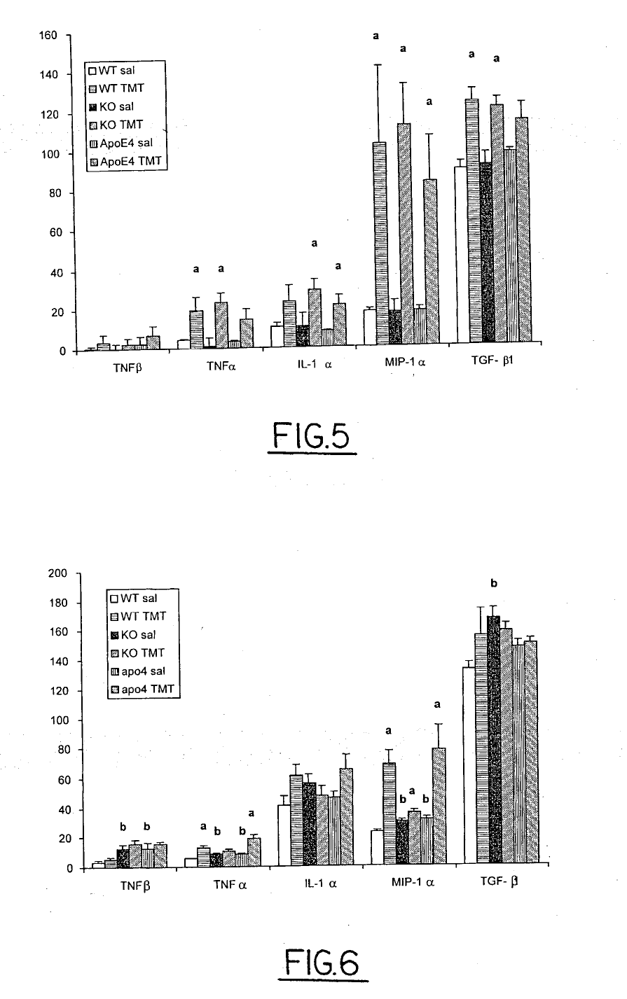

Given the proposed role for an altered immune system in the progression of AD, we examined the mRNA levels for various proinflammatory cytokines in the wild-type, APOE knockout, and APOE4 mice. At 21 days of age, examination of mRNA for TNFβ, TNFα, IL-1α, MIP-1α, and TGF-β1 demonstrated similar constitutive levels in the hippocampus of all mice examined with no difference evident between genotypes (FIG. 5). Following exposure to TMT, all mice showed elevations in mRNA levels for MIP-1α. TNFα and TGF-β1 were significantly elevated in wild-type and APOE knockout mice, while IL-1α was elevated in APOE knockout and APOE4 mice (FIG. 5).

At 8 months of age, TNFβ, TNFα, and MIP-1α mRNA levels were elevated in both the APOE knockout and APOE4 mice, and TGF-β1 in APOE knockout mice, as compared to wild-type mice (FIG. 6). No significant differences were evident in the basal levels of IL-1α mRNA. Following exposure to TMT, significant (p<0.05) elevations were seen in all animals for MIP-α. mRNA levels relative to levels in saline-injected mice (FIG. 6). However, the increase of MEP-1α mRNA levels in APOE knockout mice was lower (1.3-fold) than in wild-type (3.5-fold) or APOE4 mice (3-fold) (FIG. 6). TNFα mRNA levels were elevated (p<0.05) in both the wild-type and APOE4 mice following TMT (FIG. 6). No elevation in TNFα mRNA level was seen in APOE knockout mice. No significant elevations were seen in mRNA levels for TNFβ, IL-1α, or TGF-β1 following TMT in all animals examined.

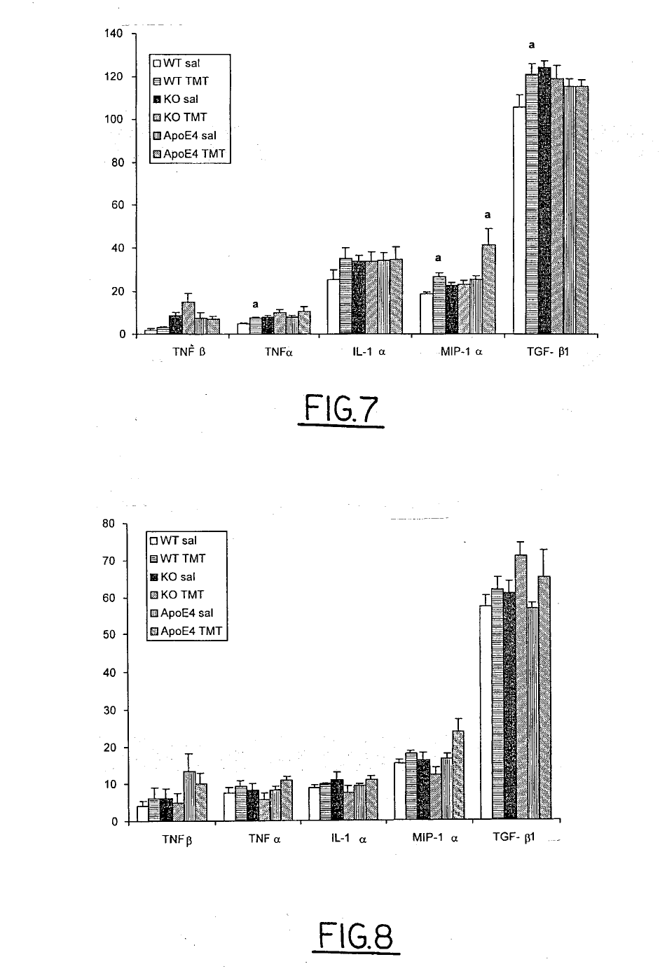

Given the genotype-specific effects in the hippocampus of 8-month-old mice following TMT, additional brain regions were examined. In the cortex, TMT exposure elevated mRNA levels for TNFα and TGF-β1 in the wild-type mice with no elevation seen in the APOE knockout or APOE4 mice (FIG. 7). MIP-1α mRNA levels in both wild-type and APOE4 mice were elevated by TMT with no elevation in APOE knockout mice (FIG. 7). In the cerebellum, TMT exposure showed no effect on mRNA levels for TNFβ, TNFα, IL-1α, MIP-1α, and TGF-β1 (FIG. 8).

The current study suggests a regulatory role for apoE in maintaining a critical balance of various proinflammatory cytokines. This was evident by the differences in basal levels of TNFβ, TNFα, MIP-1α, and TGF-β1 mRNA in the 8-month-old APOE knockout and the APOE4 mice. The shift seen with both APOE genotypes may represent altered systems that are unable to properly respond to injury. This is supported by the apoE genotype dependent influence on EB-22, TNFα, and MIP-1α mRNA levels in response to chemically induced injury.

APOE knockout mice have been reported to have increased sensitivity to brain injury (Chen et al., 1997; Laskowitz et al., 1997; Lomnitski et al., 1997; Horsburgh et al., 1997; Sheng et al., 1999). A recent in vivo study demonstrated an impaired microglia/macrophage response in APOE-deficient mice within 7 days of a cortical ischemia injury (Trieu & Uckun, 2000). These authors concluded that it may be due to the inability of microglia to remove the degeneration products of the lesion, thus creating an aversive and toxic extraneuronal environment. In the current study, the lack of an induction of TNFα, IL-1α, and MIP-1α by TMT in the APOE knockout mice may indeed reflect impairment of the microglia response. However, it also suggests that in the absence of the apoE protein, a lower level of cytokines secreted from glial cells at the site of injury would minimize the area of injury. In the early stages of injury, a decrease in cytokine/chemokine factors may serve to maintain the injury to a focal site, preventing widespread damage. While, in the later stages when microglia activation is critical to remove neuronal debris, the lack of such activation due to the loss of apoE would result in more severe focal damage. Thus, the regulation of injury-induced microglia cytokine response by apoE may play a contributing role in the progression and severity of the disease in AD patients following an acute injury or insult to the nervous system. This hypothesis is supported by the elevation of EB22 in both the APOE knockout and the APOE4 mice following-TMT. EB-22 is the murine homolog to human α-1-antichymotrypsin (ACT). It is a member of the serine-protease inhibitor family and considered to be a primary acute-phase response molecule (Inglis et al., 1991) associated with astrogliosis (Campbell et al., 1994) and amyloid deposits (Abraham et al., 1988). Thus, it has been proposed to play a role in the pathogenesis of neurodegeneration associated with AD. While the current study did not show an increase in the constitutive level of EB22 mRNA in the APOE knockout mice as has been previously reported (Licastro et al., 1999), it did demonstrate an apoE genotype dependent injury response with age, supporting a role for apoE in regulating EB22.

The lack of an injury-induced response in TGF-β1 in both the APOE knockout and the APOE4 mice suggest an influence of apoE on the response of surviving neurons. Studies of cellular localization of TGF-β1 mRNA following injury to the brain have identified both microglia (Lefebvre et al., 1992; Delree et al., 1993; Finsen et al., 1993; Morgan et al., 1993; Rogister et al., 1993) and neurons (Nichols et al., 1991; Kiefer et al., 1995) as cellular sources. Following TMT exposure, in situ hybridization localized TGF-β1 mRNA to neurons of the pyramidal cell layer (Bruccoleri et al., 1998). Based upon earlier reports of neuronal localization in models of nerve deafferentation, this localization was interpreted as a neuronal response to loss of synaptic input from mossy fibers of the dentate granule cells or a synergistic signaling interaction between the neurons and glia for pyramidal cell survival. Since the predominant response is localized to the pyramidal neurons in wild-type animals following TMT, the lack of an induction of TGF-β1 in APOE knockout mice or those containing the specific E4 allele suggest a deficiency in a neuronal protective response. The higher constitutive level of TGF-β1 in the older APOE knockout and APOE4 mice may represent either an overall increase in microglia in the brain or a generalized increase in neuronal effort for survival. These data suggest that any alterations in the APOE genotype can influence the neuronal response to injury and such alterations may contribute to a relative decrease in the survival response of neurons with age.

Accordingly, it has been shown herein that the APOE knockout mice at 21 days had close to zero basal levels of TNFβ and only minor levels of TNFα, but both TNFβ and TNFα were induced with injury. At 8 months, the basal levels were the same, but TNFβ and TNFα increases were induced by injury; a similar increase in TNFβ was induced by injury in the APOE4 transgene mice.

1. A method of providing the level of TNFβ produced by a human subject in response to tissue injury, including the steps of:
providing a sample of biological material from said subject;
assaying said sample for the presence of APOE;
on the basis of said assay, estimating the level of increase of TNFβ in said subject in response to tissue injury.

2. A method of determining the increase of TNFβ levels produced by a human subject in response to a tissue injury, including the steps of:
providing a sample of biological material from said subject;
assaying said sample for the presence of APOE; and
estimating the level of increase of TNFβ in said subject by comparing said TNFP level of said subject to the TNFβ level of a second subject that has not suffered tissue injury.

3. A method of predicting the level of TNFβ produced by a human subject following a tissue injury, including the steps of:
determining the age of the subject;
assaying a sample of biological material from said subject for the presence of APOE; and
estimating the level of TNFβ produced by the subject exposed to the tissue injury based on the age of the subject and the presence of APOE.
